(19) United States
(12) Patent Application Publication
(10) Pub. No.: US 2023/0334115 A1
(43) Pub. Date: Oct. 19, 2023

(54) METHODS AND SYSTEMS FOR SOLVING A WEIGHTED MAXIMUM CLIQUE PROBLEM

(71) Applicants: 1QB Information Technologies Inc., Vancouver (CA); Nippon Telegraph and Telephone Corporation, Tokyo (JP)

(72) Inventors: Ugur YILDIZ, Surrey (CA); Kensuke INABA, Tokyo (JP); Pooya RONAGH, Guelph (CA); Artur SCHERER, Cochrane (CA); Farhad KHOSRAVI, Edmonton (CA); Pooja PANDEY, Surrey (CA)

(21) Appl. No.: 18/331,030

(22) Filed: Jun. 7, 2023

Related U.S. Application Data
(63) Continuation of application No. PCT/IB2021/061527, filed on Dec. 9, 2021.
(60) Provisional application No. 63/123,645, filed on Dec. 10, 2020.

(51) Int. Cl. G06F 17/11; G06F 17/16

(52) U.S. Cl. CPC G06F 17/11; G06F 17/16

(57) ABSTRACT

The present disclosure provides methods and systems for solving problems. Examples of problems include, but are not limited to, maximum clique problems.

### CROSS-REFERENCE

This application is a continuation of International Application No. PCT/IB2021/061527, filed Dec. 9, 2021, which claims the benefit of U.S. Provisional Application Ser. No. 63/123,645, filed on Dec. 10, 2020, which applications are incorporated herein by reference for all purposes.

### BACKGROUND

Solving the maximum clique problem or maximum independent set problem in graph theory can be important in many theoretical and real-world applications in various fields such as chemistry and data mining, for example, molecular similarity and applications in massive data sets.

In an application, the measurement of structural similarity among molecules can play an important role in chemical and pharmaceutical research in areas such as drug discovery. The similarity can be measured based on a graph representation of the labelled molecular atom bond structures. In some cases, this is an application of the maximum clique problem.

In an example application, a graph may have certain attributes that are represented by its vertices and edges. Understanding the graph's properties may be important to analyze the system under various considerations. Call graphs and internet/web graphs are just a few examples that may be listed. Examples of such graphs can be found in Butenko, S. and Pardalos, P. M., “Maximum Independent Set and Related Problems, with Applications,” Diss. University of Florida, 2003, the disclosure of which is incorporated by reference in its entirety.

### SUMMARY

The maximum clique problem and the maximum independent set problem may be structurally similar. Since these problems are NP-hard, it may not be possible to solve them in polynomial time unless P=NP. Therefore, the development of methods and systems for solving these problems may be of importance. The time required to solve these problems can increase exponentially with the size of the problem. Methods for solving these problems at large sizes may involve heuristics, such as generic heuristics like simulated annealing and tabu search, and native heuristics like local search with strong configuration checking (LSCC). One of the disadvantages of these heuristics may be that they comprise one or many sequential processing steps that may not be suitable for parallel processing and therefore they may not take full advantage of high-performance computing (HPC) devices' capabilities.

Recognized herein is the need for improved methods and systems that can overcome at least one of the above-identified drawbacks.

The present disclosure may be directed to methods and systems for solving a weighted maximum clique problem using at least one of a matrix multiplication device and a coherent optical network. The present disclosure provides various non-limiting advantages. For example, an advantage of the methods and systems disclosed herein is that they may be highly parallelizable, allowing the use of a matrix multiplication devices to both accelerate the computations involved in each processing step and to solve multiple trials simultaneously. For example, an advantage of said methods and systems is that they may be implemented on a coherent optical network. In more detail, an advantage of one or more embodiments of the methods and systems disclosed herein is that unlike currently existing methods that cannot take full advantage of high-performance computing (HPC) devices, the methods and systems disclosed herein can be easily parallelized for multiple trials.

According to an aspect of the present disclosure, there is disclosed a method for solving a weighted maximum clique problem using at least one of a matrix multiplication device and a coherent optical network, the method comprising obtaining an indication of a weighted maximum clique problem; formulating said problem as a quadratic continuous optimization problem with one simplex constraint; constructing a stochastic differential equation corresponding to said formulated optimization problem comprising a plurality of continuous variables; using at least one of a matrix multiplication device and a coherent optical network to solve said stochastic differential equation at least one time; obtaining at least one solution of said stochastic differential equation; using said at least one solution of said stochastic differential equation to obtain at least one solution for said maximum clique problem.

In accordance with one or more embodiments, the method further comprises calculating at least one stochastic gradient and updating said plurality of said continuous variables.

In accordance with one or more embodiments, the method further comprises selecting a highest clique value.

In accordance with one or more embodiments, the matrix multiplication device comprises at least one member of the group consisting of a graphics processing unit (GPU), a tensor processing unit (TPU), a field-programmable gate array (FPGA), and an application-specific integrated circuit (ASIC), and a tensor streaming processor (TSP).

In accordance with one or more embodiments, the method further comprises using said stochastic differential equation to configure said coherent optical network.

In accordance with one or more embodiments, the method for solving a weighted maximum clique problem is used for solving a graph similarity problem.

In accordance with one or more embodiments, the method for solving a weighted maximum clique problem is used for solving a molecular similarity problem.

According to an aspect of the present disclosure, there is disclosed a system for solving weighted maximum clique problem using a matrix multiplication device or a coherent optical network, the system comprising a digital computer comprising an interface and a non-transitory computer readable medium operatively coupled to a processor, said non-transitory computer readable medium comprising instructions, wherein said processor is configured to execute said instructions to at least obtain an indication of a weighted maximum clique problem; formulate said problem as a quadratic continuous optimization problem with one simplex constraint; construct a stochastic differential equation corresponding to the formulated optimization problem comprising a plurality of continuous variables; a memory operatively coupled to said digital computer, wherein said memory stores at least said solution of the stochastic differential equation and at least one said solution for the maximum clique problem; and a computational platform operatively coupled to said digital computer, said computational platform comprising at least one processor and a readout control system, wherein said computational platform is configured at least to receive from said digital computer an indication of said stochastic differential equation, solve said stochastic differential equation to generate a solution, and via said readout control, store said solution of said stochastic differential equation in said memory.

In accordance with one or more embodiments, the computational platform comprises at least one member selected from the group consisting of a field-programmable gate array (FPGA), an application-specific integrated circuit (ASIC), a central processing unit (CPU), a graphics processing unit (GPU), a tensor processing unit (TPU), a tensor streaming processor (TSP), and a coherent optical network.

In accordance with one or more embodiments, the one or more processors of the at least one processor is located on a cloud.

In accordance with one or more embodiments, the memory is operatively coupled to the digital computer over a network.

Another aspect of the present disclosure provides a non-transitory computer readable medium comprising machine-executable code that, upon execution by one or more computer processors, implements any of the methods disclosed elsewhere herein.

Another aspect of the present disclosure provides a system comprising one or more computer processors and computer memory coupled thereto. The computer memory comprises machine-executable code that, upon execution by the one or more computer processors, implements any of the methods disclosed elsewhere herein.

Additional aspects and advantages of the present disclosure will become readily apparent to those skilled in the art from the following detailed description, wherein only illustrative embodiments of the present disclosure are shown and described. As will be realized, the present disclosure may pertain to other and different embodiments, and its several details may include modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a flowchart that shows an example of a method for solving a weighted maximum clique problem.

FIG. 2A is a diagram that shows an example of a system for solving a weighted maximum clique problem using a matrix multiplication device.

FIG. 2B is a diagram that shows an example of a system for solving a weighted maximum clique problem using a coherent optical network.

FIG. 3 is a diagram that shows an example of a vertex weighted graph and the corresponding maximum clique.

FIG. 4 is s a flowchart that shows an example of a procedure for updating the values of the variables.

FIG. 5 is a diagram that shows an example of a coherent optical network.

FIG. 6 is a diagram that shows a computer system that is programmed or otherwise configured to implement the methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “weighted graph” generally refers to a vertex weighted graph, namely a graph wherein the weights are attached to vertices.

As used herein, the term “clique” (of a graph) generally refers to a subset of vertices such that distinct vertices are adjacent.

As used herein, the term “maximum clique” (of the vertex weighted graph) generally refers to a clique where the summation of the weights attached to the vertices of the clique are increased. For example, the summation may be a highest known summation. For example, the summation of the weights may be a relative maximum or an absolute maximum.

As used herein, the term “weighted maximum clique problem” generally refers to the maximum clique problem wherein a weight can be attached to a vertex. In some cases, the weight attached may be any weight, including the case wherein all weights equal 1.

As used herein, the term “solution to the weighted maximum clique problem” generally refers to the value of the weighted maximum clique, for example, a solution to the summation of the weights of the vertices comprising the weighted maximum clique. Term “solution” may refer to an optimal solution or a sub-optimal solution.

The present disclosure provides methods and systems for solving a weighted maximum clique problem using at least one of a matrix multiplication device and a coherent optical network.

### Motzkin-Straus Model and Construction of Stochastic Differential Equation

In an example, the Ising problem whose spins can take the values of −1 or +1 are considered (see, for example, Ising, E., “Beitrag zur Theorie des Ferromagnetismus,” Z. Phys., 31:253-258 (1925), which is incorporated by reference herein for all purposes).

The Ising problem may reduce or, in some cases, minimize the system energy with respect to the couplings Jᵢⱼ between the spins i∈N and j∈N. The problem may reduce or, in some cases, minimize, the objective function E(σ) Σ_{i,j∈N}Jᵢⱼσᵢσⱼ subject to σⱼ∈{−1,1}∀j∈N.

The above quadratic unconstrained binary optimization problem may be solved using the following stochastic differential equation for each spin δσⱼ=Σ_{i∈N}Jᵢⱼσᵢ+(1−σⱼ²)σⱼ+dWⱼ, where dWⱼ is representative of noise enabling the procedure to escape local minima. Solving the stochastic differential equation above may result in having positive or negative values for the spins.

The binary constraints σⱼ∈{−1,1}∀j∈N may be represented by the second term of the stochastic differential equation.

The maximum clique problem is a combinatorial problem with discrete variables; however, Motzkin and Straus (Motzkin, T. S. and Straus, E. G., “Maxima for Graphs and a New Proof of a Theorem of Turán,” Canadian Journal of Mathematics, 17: 533-40 (1965), which is incorporated herein by reference for all purposes) introduce a quadratic continuous model of the maximum clique problem. The Motzkin-Straus model has a single simplex constraint in addition to nonnegativity constraints. The model may increase, or in some cases, maximize, the objective function g(x)=Σ_{i,j∈N}Aᵢⱼxᵢxⱼ subject to the simplex constraint Σ_{i∈N}xᵢ=1, where xᵢ≥0∀i. Once a solution is obtained, the clique value (the objective function value in this case) may be calculated using the equation

$$ω ( w , G ) = \frac{1}{1 - g ( {x}^{*} )} ,$$

where ω is the clique value for the graph and x* is a maximizer of g(x).

To construct a stochastic differential equation able to solve the Motzkin-Straus model, the variables xᵢ are replaced with cᵢ² for each variable i so that the unrestricted variables may be used to devise the methods and systems disclosed herein.

The Motzkin-Straus model may be rewritten using the variables cᵢ∀i in the new objective function g(c)=Σ_{i,j∈N}Aᵢⱼcᵢ²cⱼ² subject to the simplex constraint Σ_{i∈N}cᵢ²=1, to eliminate nonnegativity constraints. In some cases, such constraints may prevent solving using at least one of a matrix multiplication device and a coherent optical network.

Similar to the handling of the binary constraints of the Ising model above, the simplex constraint may be handled by revising the second term of the stochastic differential equation. The stochastic differential equation δcⱼ=Σ_{i∈N}Aᵢⱼcᵢ²cⱼ+(1−Σ_{i∈N}cᵢ²)cⱼ+dWⱼ may be used for each variable cⱼ to solve the Motzkin-Straus model.

### High-Performance Computing Device

A computing device herein which may interact with a photonic computing device herein, for example, a coherent optical network, may be a high-performance computing (HPC) device. An HPC may comprise one or more of a graphics processing unit (GPU), a tensor processing unit (TPU), a field-programmable gate array (FPGA), an application-specific integrated circuit (ASIC), and a tensor streaming processor (TSP). Any other suitable processing unit that is capable of performing matrix multiplication may be used. Certain computing devices may be more efficient at operations such as matrix multiplication. These computing devices may provide additional efficiency gains over other computing systems. For example, a matrix multiplication device may be a GPU. A GPU may be a specialized electronic circuit optimized for high throughput, which can perform the same set of operations in parallel on many data blocks at a time. For example, a matrix multiplication device may be a TPU. A TPU may be a type of ASIC developed for low bit precision processing by Google® Inc.; see U.S. Patent Application No. 2016/0342891, the disclosure of which is incorporated by reference in its entirety. For example, a matrix multiplication device may be an FPGA. An FPGA may be an integrated circuit chip that comprises configurable logic blocks and programmable interconnects. It can be programmed after manufacturing to execute custom algorithms. For example, a matrix multiplication device may be an ASIC. An ASIC may be an integrated circuit chip that is customized to run a specific algorithm. In some case, an ASIC cannot be programmed after manufacturing. For example, a matrix multiplication device may be a TSP. A TSP may be a domain-specific programmable integrated chip that is designed for linear algebra computations as they may be performed in artificial intelligence applications, an example of which may be found in Gwennap, L., “Groq Rocks Neural Networks,” Microprocessor Report, January 2020, which is incorporated entirely herein by reference for all purposes.

### Coherent Ising Machine

In some cases, a computing device such as a matrix multiplication device may be couple to a photonic computing device. A photonic computing device may be a coherent Ising machine (CIM). A CIM is a photonic device a powerful optimization system due to its increased, e.g., all-to-all, connectivity among spins. The coherent Ising machine may be adapted to solve the Ising problem; however, its optical pulses may instead be used to represent continuous variables. Such a modification may be also referred to as a coherent optical network.

The coherent optical network may be of various types such as any type described herein. In some cases, the coherent optical network comprises optical computing devices. In some cases, the coherent optical network comprises any or some of optical instruments such as beam splitters, free-space lasers that navigate on optical tables, fibre-ring cavities comprising optical fibres and fibre optics instruments. In some cases, the coherent optical network comprises integrated photonics.

### Optical Computing Devices

In some cases, the optical device comprises a network of optical parametric oscillators (OPOs) as disclosed in U.S. Patent Application No. 2016/0162798 and in International Patent Publication No. WO2015/006494, the disclosure of each of which is incorporated by reference in their entirety.

In some cases, each spin of the Ising model is simulated by an optical parametric oscillator (OPO) operating at degeneracy.

Degenerate optical parametric oscillators (OPO) are open dissipative systems that experience second-order phase transition at the oscillation threshold. Because of the phase-sensitive amplification, a degenerate OPO may oscillate with a phase of either 0 or π with respect to the pump phase for amplitudes above the threshold. The phase is random, affected by the quantum noise associated with the optical parametric down conversion during the oscillation build-up. Therefore, a degenerate OPO naturally represents a binary digit specified by its output phase. Based on this property, a degenerate OPO system may be used as a physical representative of an Ising spin system. The phase of each OPO is identified as an Ising spin, with its amplitude and phase determined by the strength and the sign of the Ising coupling between relevant spins.

When pumped by a strong source, a degenerate optical parametric oscillator (OPO) takes one of two phase states corresponding to spin +1 or −1 in the Ising model. A network of N substantially identical OPO with mutual coupling are pumped with the same source to simulate an Ising spin system. After a transient period from the introduction of the pump, the network of OPOs approaches a steady state.

In some cases, wherein the amplitudes are above the threshold but a degenerate optical parametric oscillator is pumped by a weak source, it may represent a continuous variable instead of the binary spin.

The phase state selection process depends on the vacuum fluctuations and mutual coupling of the optical parametric oscillators (OPO). In some cases, the pump is pulsed at a constant amplitude, in other implementations the pump output is gradually increased, and in yet further implementations, the pump is controlled in other ways.

In some examples of an optical device, the plurality of couplings of the Ising model are simulated by a plurality of configurable couplings used for coupling the optical fields between optical parametric oscillators (OPO). The configurable couplings may be configured to be off or configured to be on. Turning the couplings on and off may be performed gradually or abruptly. When configured to be on, the configuration may provide any phase or amplitude depending on the coupling strengths of the Ising model.

Each optical parametric oscillator (OPO) output may be interfered with a phase reference and the result is captured at a photodetector. The OPO outputs represent a configuration of the Ising model. For example, a zero phase may represent a spin −1 state, and a π phase may represent a +1 spin state in the Ising model.

For the Ising model with spins, and according to some examples, a resonant cavity of the plurality of optical parametric oscillators (OPO) is configured to have a round-trip time equal to times the period of pulses from a pump source. Round-trip time as used herein indicates the time for light to propagate along one pass of a described recursive path. The pulses of a pulse train with period equal to the period of the resonator cavity round-trip time may propagate through the OPOs concurrently without interfering with each other.

In some cases, the couplings of the optical parametric oscillators (OPO) are provided by a plurality of delay lines allocated along the resonator cavity.

The plurality of delay lines comprises a plurality of modulators which synchronously control the strengths and phases of couplings, allowing for programming of the optical device to simulate the Ising model.

In a network of optical parametric oscillators (OPO), delay lines and corresponding modulators are enough to control amplitude and phase of coupling between every two OPOs.

In some cases, a device capable of sampling from an Ising model can be manufactured as network of optical parametric oscillators (OPO) as disclosed in U.S. Patent Application No. 2016/0162798, the disclosure of which is incorporated by reference in its entirety.

In some cases, the network of optical parametric oscillators (OPO) and couplings of OPOs are achieved using commercially available mode locked lasers and optical elements, such as telecom fiber delay lines, modulators, and other optical devices. Alternatively, the network of OPOs and couplings of OPOs are implemented using optical fiber technologies, such as fiber technologies developed for telecommunications applications. In some cases, the couplings may be realized with fibers and controlled by optical Kerr shutters.

### Integrated Photonic Coherent Ising Machine

Another example of an analogue system capable of performing sampling from low-energy states of an Ising model near its equilibrium state is an integrated photonic coherent Ising machine disclosed for instance in Kako, S., et al., “Coherent Ising Machines with Error Correction Feedback” by Kako, S., et al., Adv. Quantum Technol. 3:2000045 (2020), the disclosure of which is incorporated by reference in its entirety.

In some cases, an integrated photonic coherent Ising machine is a combination of nodes and a connection network solving a particular Ising problem. In some cases, the combination of nodes and the connection network may form an optical computer that is adiabatic. In other words, the combination of the nodes and the connection network may non-deterministically solve an Ising problem when the values stored in the nodes reach a steady state to minimize the energy of the nodes and the connection network. Values stored in the nodes at the minimum energy level may be associated with values that solve a particular Ising problem.

In some cases, a system comprises a plurality of ring resonator photonic nodes, wherein each one of the plurality of ring resonator photonic nodes stores a value; a pump coupled to each one of the plurality of ring resonator photonic nodes via a pump waveguide for providing energy to each one of the plurality of ring resonator photonic nodes; and a connection network comprising a plurality of two by two building block of elements, wherein each element of the two by two building block comprises a plurality of phase shifters for tuning the connection network with parameters associated with encoding of an Ising problem, wherein the connection network processes the value stored in the each one of the plurality of ring resonator photonic nodes, wherein the Ising problem is solved by the value stored in the each one of the plurality of ring resonator photonic nodes at a minimum energy level.

### Digital Computer

In some cases, the digital computer comprises one or more hardware central processing units (CPUs) that carry out the digital computer's functions. In some cases, the digital computer further comprises an operating system configured to perform executable instructions. In some cases, the digital computer is connected to a computer network. In some cases, the digital computer is connected to the Internet such that it accesses the World Wide Web. In some cases, the digital computer is connected to a cloud computing infrastructure. In some cases, the digital computer is connected to an intranet. In some cases, the digital computer is connected to a data storage device.

Various types of digital computer may be used. In fact, suitable digital computers may include, by way of non-limiting examples, server computers, desktop computers, laptop computers, notebook computers, sub-notebook computers, netbook computers, netbook computers, set-top computers, media streaming devices, handheld computers, Internet appliances, mobile smartphones, tablet computers, personal digital assistants, video game consoles, and vehicles. Smartphones may be suitable for use with one or more examples of the method and the system described herein. Select televisions, video players, and digital music players, in some cases with computer network connectivity, may be suitable for use in some cases of the system and the method described herein. Suitable tablet computers may include those with booklet, slate, and convertible configurations.

In some cases, the digital computer comprises an operating system configured to perform executable instructions. The operating system may be, for example, software, comprising programs and data, which manages the device's hardware and provides services for execution of applications. Various types of operating system may be used. For example, suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some cases, the operating system is provided by cloud computing. Suitable mobile smart phone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Suitable media streaming device operating systems may include, by way of non-limiting examples, Apple TV®, Roku®, Boxee®, Google TV®, Google Chromecast®, Amazon Fire®, and Samsung® HomeSync®. Suitable video game console operating systems may include, by way of non-limiting examples, Sony® PS3@, Sony® PS4@, Microsoft® Xbox 360@, Microsoft® Xbox One®, Nintendo® Wii®, Nintendo® Wii U®, and Ouya®.

In some cases, the digital computer comprises a storage and/or memory device. Various types of storage and/or memory may be used in the digital computer. In some cases, the storage and/or memory device comprises one or more physical apparatuses used to store data or programs on a temporary or permanent basis. In some cases, the device comprises a volatile memory and requires power to maintain stored information. In some cases, the device comprises non-volatile memory and retains stored information when the digital computer is not powered. In some cases, the non-volatile memory comprises a flash memory. In some cases, the non-volatile memory comprises a dynamic random-access memory (DRAM). In some cases, the non-volatile memory comprises a ferroelectric random access memory (FRAM). In some cases, the non-volatile memory comprises a phase-change random access memory (PRAM). In some cases, the device comprises a storage device including, by way of non-limiting examples, CD-ROMs, DVDs, flash memory devices, magnetic disk drives, magnetic tapes drives, optical disk drives, and cloud computing based storage. In some cases, the storage and/or memory device comprises a combination of devices, such as those disclosed herein.

In some cases, the digital computer comprises a display used for providing visual information to a user. Various types of display may be used. In some cases, the display comprises a cathode ray tube (CRT). In some cases, the display comprises a liquid crystal display (LCD). In some cases, the display comprises a thin film transistor liquid crystal display (TFT-LCD). In some cases, the display comprises an organic light-emitting diode (OLED) display. In some cases, an OLED display comprises a passive-matrix OLED (PMOLED) or active-matrix OLED (AMOLED) display. In some cases, the display comprises a plasma display. In some cases, the display comprises a video projector. In some cases, the display comprises a combination of devices, such as those disclosed herein.

In some cases, the digital computer comprises an input device to receive information from a user. Various types of input devices may be used. In some cases, the input device comprises a keyboard. In some cases, the input device comprises a pointing device including, by way of non-limiting examples, a mouse, trackball, trackpad, joystick, game controller, or stylus. In some cases, the input device comprises a touch screen or a multi-touch screen. In some cases, the input device comprises a microphone to capture voice or other sound input. In some cases, the input device comprises a video camera or other sensor to capture motion or visual input. In some cases, the input device comprises a Kinect™, Leap Motion™, or the like. In some cases, the input device comprises a combination of devices, such as those disclosed herein.

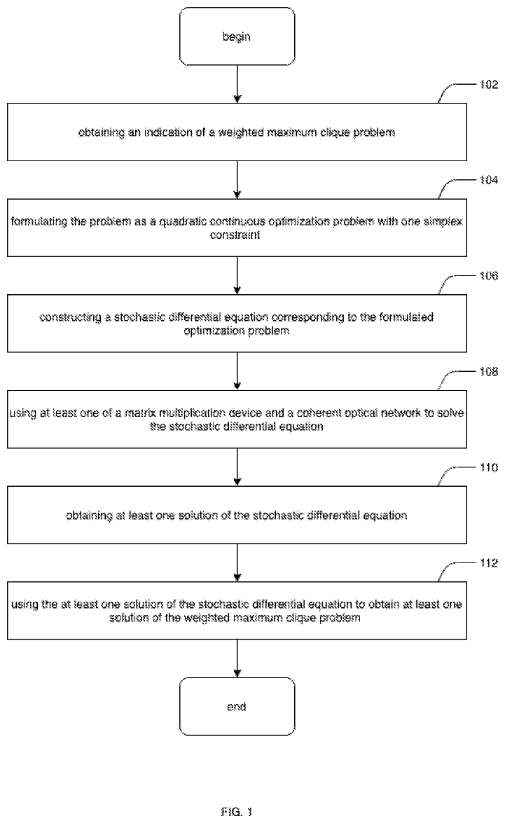

Now referring to FIG. 1, there is shown a flowchart of an example of a method for solving a weighted maximum clique problem. More precisely and in some cases, the method comprises using at least one of a matrix multiplication device and a coherent optical network.

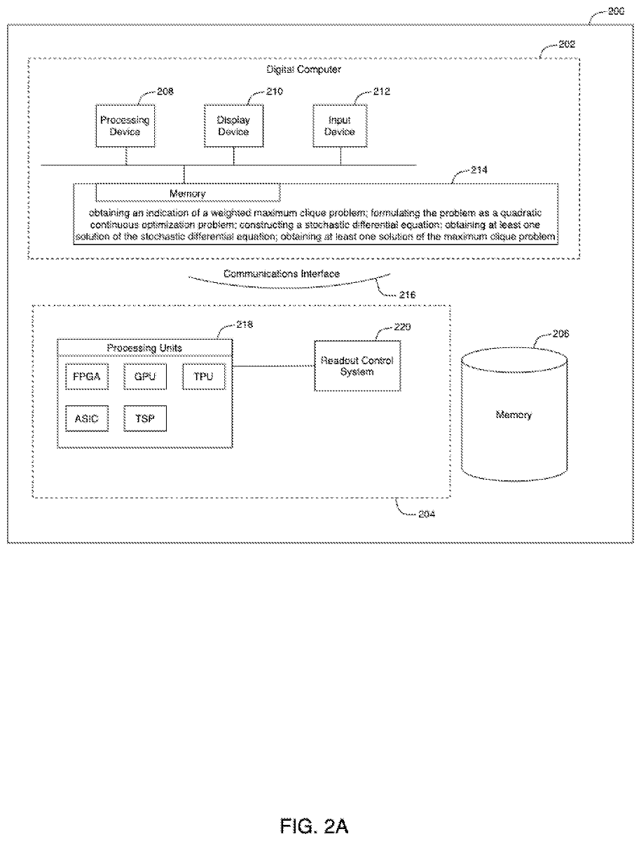

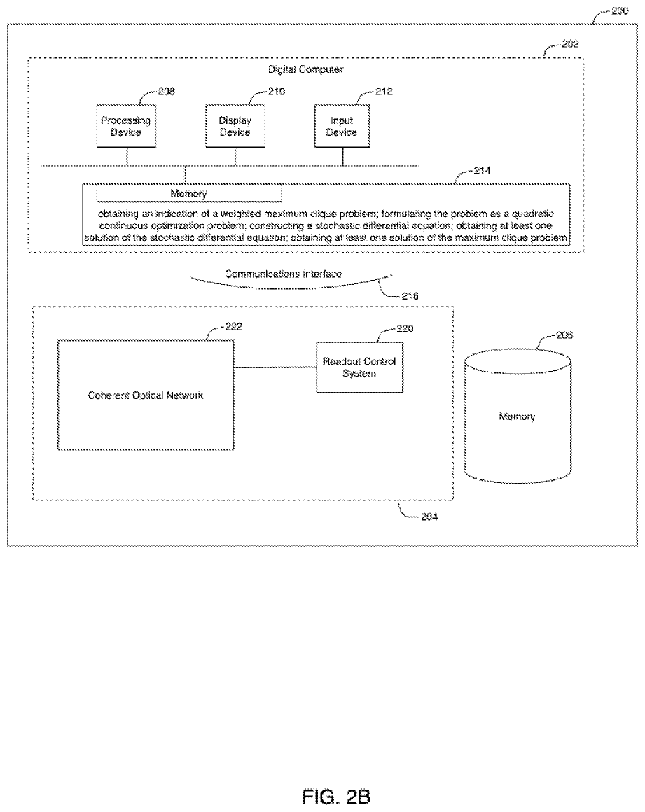

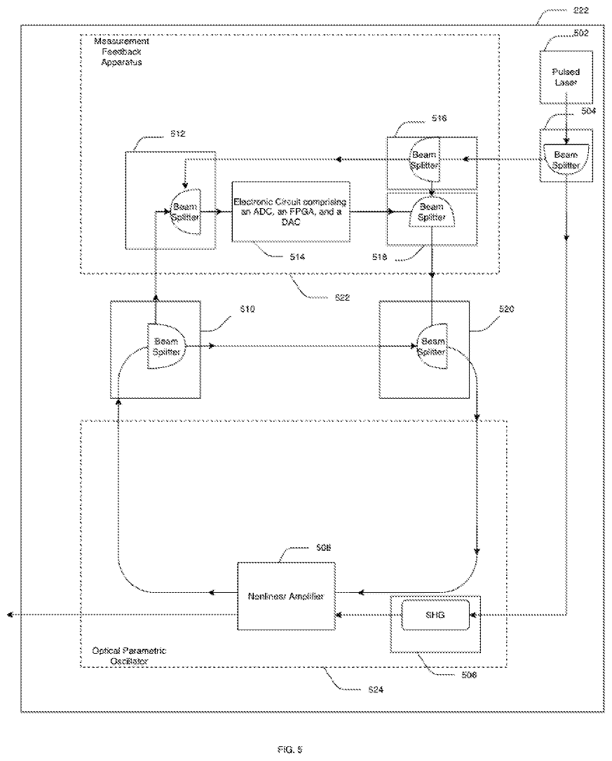

At least one matrix multiplication device may be any suitable matrix multiplication device, such as any matrix multiplication device described herein with respect to FIG. 2. The coherent optical network may be any suitable coherent optical network, such as any coherent optical network described herein with respect to FIG. 2 and/or FIG. 5.

Still referring to FIG. 1 and according to processing operation 102, an indication of a weighted maximum clique problem is obtained. In some cases, the indication of a weighted maximum clique problem may be obtained in various ways. For example, the indication of a weighted maximum clique problem is obtained from a user.

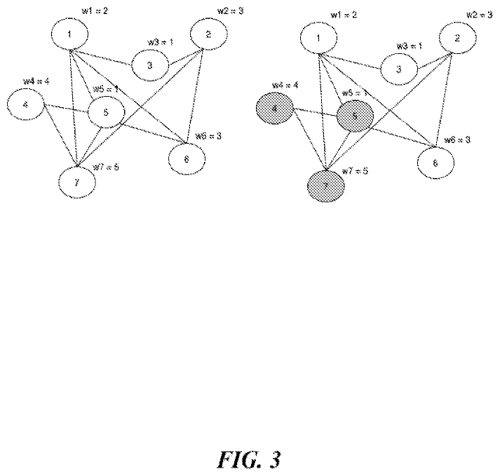

The weighted maximum clique problem may be defined in the following way. Given a vertex weighted graph G, the weighted maximum clique problem seeks to find a complete subgraph that increases, e.g., maximizes, the summation of the weights of the nodes of the complete subgraph. Now referring to FIG. 3, there is shown an example of a vertex weighted graph and the corresponding weighted maximum clique. The weighted maximum clique in this example comprises vertices 4, 5 and 7 and the corresponding optimal objective function value is 10. It is calculated using the weights associated with the vertices composing the weighted maximum clique.

Still referring to FIG. 1 and according to processing operation 104 the weighted maximum clique problem is formulated as a quadratic continuous optimization problem with one simplex constraint.

The weighted maximum clique problem can be formulated in various ways; see, for example, Pardalos, P. M. and Xue, J., “The Maximum Clique Problem,” J. Global Optimization, 4.3: 301-328 (1994), the disclosure of which is incorporated by reference in its entirety. In the case wherein all the weights of the graph equal 1, in other words in the case of an unweighted graph, the Motzkin-Straus approach (Motzkin, T. S. and Straus, E. G., “Maxima for Graphs and a New Proof of Theorem of Turin,” Canadian Journal of Mathematics, 17: 533-40 (1965), the disclosure of which is incorporated by reference in its entirety) comprising formulating the maximum clique problem as a quadratic continuous optimization problem may be used to solve the maximum clique problem. The Motzkin-Straus model may be generalized to solve the maximum clique problem of a weighted graph comprising at least one weight not equal to 1; see, for example, Gibbons, L. E., et al., “Continuous Characterizations of the Maximum Clique Problem,” Mathematics of Operations Research, 22(3):754 (1997), which is incorporated herein by reference for all purposes. In some cases, the weighted maximum clique problem refers to the case wherein any weight can be attached to a vertex, including the case wherein all weights equal 1. In some examples, the Motzkin-Straus model or its generalization to weighted graphs is used to solve a weighted maximum clique problem using a high-performance computing (HPC) device or coherent optical network. The HPC device may comprise one or more of a graphics processing unit (GPU), a tensor processing unit (TPU), a field-programmable gate array (FPGA), an application-specific integrated circuit (ASIC), and a tensor streaming processor (TSP). Other suitable processing units that are capable of performing matrix multiplication may be used such as any processing unit described herein with respect to FIG. 2.

Given a vertex weighted graph G=(V, E, w), the class of symmetric n×n matrices defined as

$$M ( w , G ) = \{ B = {( {b}_{ij} )}_{i , j ∈ V} : {b}_{ii} = \frac{1}{{w}_{i}} , ∀ i , \\ {b}_{ij} + {b}_{ji} \geq \frac{1}{{w}_{i}} + \frac{1}{{w}_{j}} if ( i , j ) ∉ E , {b}_{ij} = 0 , otherwise \}$$

is considered and the quadratic continuous programming problem, which solves maximum clique problem, is defined as min f(x)=Σ_{∀(i,j)∈E}bᵢⱼxᵢxⱼ subject to Σ_{∀i∈V}xᵢ−1 and xᵢ≥0∀i∈V, where B∈M(w, G). Once the above problem is solved, the maximum clique value is defined by

$$ω ( w , G ) = \frac{1}{f ( {x}^{*} )} ,$$

where x* is a minimizer of f(x).

Still referring to FIG. 1 and according to processing operation 106, a stochastic differential equation corresponding to the formulated optimization problem is constructed.

Some ideas of the Motzkin-Straus model may be applied to obtain a differential equation that solves the maximum clique problem of a weighted graph.

Herein the variables may take any nonnegative continuous values. The variables defined via xⱼ=cⱼ² where cⱼ have an unrestricted spin j∈V. The quadratic continuous optimization problem min f(x)=Σ_{∀(i,j)∉E}Bᵢⱼcᵢ²cⱼ² subject to Σ_{∀i∈V}cⱼ²=1 and cⱼ²≥0∀j∈V is considered.

The following stochastic differential equations defined for each variable solve the quadratic continuous optimization problem defined above: δcⱼ=Σ_{∀(i,j)∉E}Bᵢⱼcᵢ²cⱼ−(1−Σᵢ cᵢ²)cⱼ+dWⱼ.

Similar to the stochastic differential equation described in the Motzkin-Straus model, the second part of the stochastic differential equation constructed in the continuous case herein penalizes for the constraint Σ_{∀i∈V}cⱼ²=1, and Wⱼ is the Wiener process representative of the noise term in a corresponding Langevin equation. The Wiener process is simulated using samples from Gaussian distributions and enables the method to escape local minima. In some cases, wherein a coherent optical network is used to solve the weighted maximum clique problem, the noise term models the quantum noise afflicting optical pulses.

Still referring to FIG. 1 and according to processing operation 108, the stochastic differential equation is solved. The stochastic differential equation may be solved using a matrix multiplication device. The matrix multiplication device may be any suitable matrix multiplication device, such as any matrix multiplication device described herein with respect to FIG. 2. The matrix multiplication device may comprise a high-performance computing (HPC) device. The HPC device may be of various types such as any HPC device described elsewhere herein. The HPC device may comprise one or more of a graphics processing unit (GPU), a tensor processing unit (TPU), a field-programmable gate array (FPGA), an application-specific integrated circuit (ASIC), and a tensor streaming processor (TSP). Any other suitable processing unit that is capable of performing matrix multiplication may be used such as any processing unit described herein with respect to FIG. 2.

The stochastic differential equation may be solved using a coherent optical network. The coherent optical network may be any suitable coherent optical network, such as any coherent optical network described herein with respect to FIG. 2 and/or FIG. 5. The coherent optical network may comprise one or more of an optical computing device and an integrated photonics coherent Ising machine.

Solving the stochastic differential equation may comprise providing an indication of the stochastic differential equation to the at least one of a matrix multiplication device and a coherent optical network. The indication of a stochastic differential equation may be provided according to various examples. In some cases, the indication of the stochastic differential equation is obtained by a communications interface 216 of a computing system 200 via a data network. In some cases, the indication of the stochastic differential equation is transmitted by a digital computer 202 operatively connected to the communications interface 216 via the data network. The digital computer 202 may be any suitable digital computer, such as any digital computer described herein with respect to the system 200 disclosed in FIG. 2A and FIG. 2B.

Moreover, the communication interface 216 may be of various types. In some cases, the communications interface comprises an application programming interface (API) gateway.

In some cases, wherein a matrix multiplication device is used, the stochastic differential equation is solved using Monte Carlo simulations in which values assigned to variables are updated by sampling stochastic gradients. In some cases, the stochastic gradients are unbiased estimators of average gradients that coincide with the gradients of the objective functions in the stochastic differential equation and its corresponding Langevin dynamics.

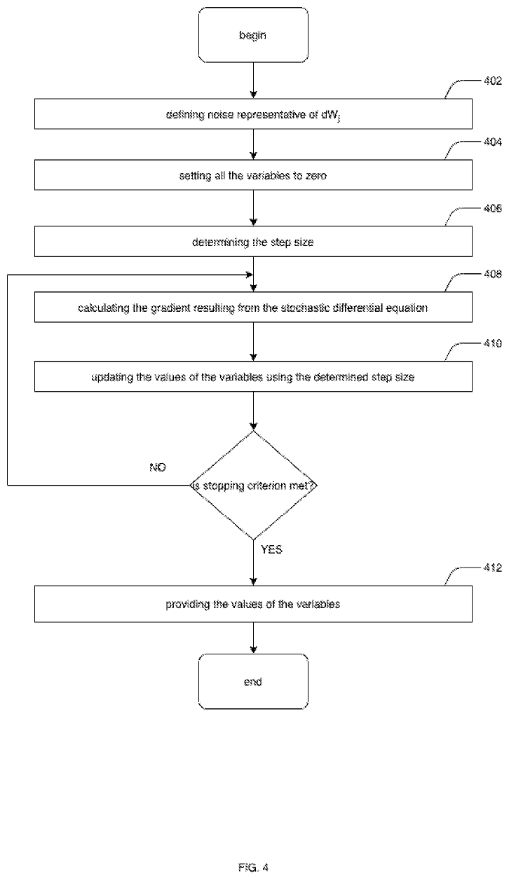

Now referring to FIG. 4, there is shown a flowchart of an example of the update procedure for the variables' assigned values using stochastic gradients.

According to processing operation 402 the noise may be defined. The noise is distributed according to a Gaussian distribution dWⱼ with parameters (μⱼ, σⱼ), where the parameters may be assigned various values. In some cases, the parameters are assigned the values μ=0 and σ=0.005. dWⱼ is representative of noise in the stochastic differential equation.

Still referring to FIG. 4 and according to processing operation 404, in some cases, the variables may be set to zero at the beginning of the procedure. In some cases, the variables may be initialized stochastically. In some cases, the stochastic initialization of variables is performed according to Gaussian distributions with mean 0 and small standard deviations.

According to processing operation 406, a step size is determined. The step size may have various values. In some cases, the step size equals 0.01 (α=0.01). In some cases, the step size is constant and determined by a user. In some cases, the step size may change according to a schedule. In some cases, a step size schedule is given by hyperparameters of the schedule determined by a user. In some cases, the step size is adaptively changed via a heuristic. In some cases, the step size is adaptively changed according to historical values of the partial derivatives corresponding to the stochastic gradients. In some embodiments, the historical values comprise a moving window of historical values.

According to processing operation 408, the stochastic gradients corresponding to the stochastic differential equation are calculated. The calculation of stochastic gradients comprises calculating a partial derivative stochastically sampled for each variable. The partial derivative with respect to the variables cⱼ, j∈V, is calculated as follows: δcⱼ=Σ_{∀(i,j)∉E}Bᵢⱼcᵢ²cⱼ−(1−Σᵢ cᵢ²)cⱼ+dW(μ, σ). In some cases, the partial derivative calculation comprises at least one matrix multiplication.

According to processing operation 410, for each variable the value is updated using the equation cⱼ=cⱼ−αδcⱼ, wherein a is the determined step size. The update calculation may comprise matrix multiplication.

If the stopping criterion is met, the variables' values are provided according to processing operation 412. The stopping criterion may be of various types. In some cases, the stopping criterion is met after the variables' assigned values are updated a certain number of times. In some cases, the stopping criterion is met when the new variable assignments are within a certain proximity of the previous values. In some cases, this proximity is represented by hyperparameters provided by the user.

If the stopping criterion is not met, processing operations 408 and 410 are repeated.

In some cases, the variables' update procedure such as the variables' update procedure described herein may be repeated a number of times to improve the probability of obtaining the optimal solution. The repetitions of the procedure may occur in parallel, taking advantage of the high-performance computing (HPC) device's capabilities. In some cases, the process is further parallelized by performing independent batches of the Monte Carlo simulation of the stochastic differential equation in parallel. In some embodiments the batch size for the number of repetitions may vary. In some cases, the batch size is a hyperparameter defined by a user.

Now referring back to FIG. 1, in some cases, wherein a coherent optical network is used in processing operation 108, the stochastic differential equation is solved via allowing the system to approach a steady state to reduce, e.g., minimize the energy. In some cases, the coherent optical network is configured using the stochastic differential equation.

Still referring to FIG. 1 and according to processing operation 110, at least one solution of the stochastic differential equation is provided. The at least one solution of the stochastic differential equation may be provided according to various embodiments. In some cases, the at least one solution of the stochastic differential equation is provided by a communications interface 216 of a system via a data network. In some cases, the at least one solution of the stochastic differential equation is transmitted by a digital computer 202 operatively connected to the communications interface 216 via the data network. In some cases, the digital computer may be any suitable digital computer, such as any digital computer described herein with respect to the system 200 disclosed in FIG. 2A and FIG. 2B.

The communications interface 216 may be of various types. In some cases, the communications interface comprises an API gateway.

Still referring to FIG. 1 and according to processing operation 112, the maximum clique's value is obtained using the at least one provided solution of the stochastic differential equation and the formula

$$ω ( w , G ) = \frac{1}{f ( {c}^{*} )} = \frac{1}{{\sum }_{∀ ( i , j ) ∉ E} {b}_{ij} {c}_{i}^{* 2} {c}_{j}^{* 2}} .$$

The term “solution” may refer to an optimal solution or a sub-optimal solution.

Now referring to FIG. 2 there is shown a diagram of an example of a system for solving a weighted maximum clique problem. FIG. 2A may be a diagram of an example of a system for solving a weighted maximum clique problem using a matrix multiplication device, and FIG. 2B is a diagram of an example of a system for solving a weighted maximum clique problem using a coherent optical network.

The system 200 comprises a digital computer 202 comprising a processing device 208 and a memory 214 comprising a computer program executable by the processing device to obtain an indication of a weighted maximum clique problem; to formulate the problem as a quadratic continuous optimization problem; to construct a stochastic differential equation; to obtain at least one solution of the stochastic differential equation; to obtain a solution to the weighted maximum clique problem.

The digital computer 202 may be of various types such as, for example, the types of digital computer as disclosed elsewhere herein.

In the example disclosed in FIG. 2A, the system 200 further comprises a computational platform 204. The computational platform 204 may comprise at least one processing unit 218 comprising a matrix multiplication device. The at least one processing unit 218 may be of various types of processors such as, for example, the types of processors as described elsewhere herein. For example, the at least one processor can comprise at least one or more of a field-programmable gate array (FPGA), an application-specific integrated circuit (ASIC), a graphics processing unit (GPU), a tensor processing unit (TPU), and a tensor streaming processor (TSP). Other suitable processing units that are capable of performing matrix multiplication may be used such as any processing unit described herein with respect to FIG. 2.

Each component of the system (e.g., the hardware) may be used as part of the system to execute a whole method, or any portion thereof, alone or in combination with other components (e.g., other hardware). In some cases, the components may be used for defining dWⱼ which is representative of noise, calculating the stochastic gradients resulting from the stochastic differential equation, determining the step size, setting all the variables to zero, updating the variable assignments using the determined step size, providing the variables' assigned values including a part or all of the above.

In FIG. 2A, the processing unit 218 may comprise one or more virtual machines. The one or more virtual machines may be one or more emulations of one or more computer systems. The virtual machines may be process virtual machines (e.g., virtual machines configured to implement a process in a platform-independent environment). The virtual machines may be systems virtual machines (e.g., virtual machines configured to execute an operating system and related programs). The virtual machine may be configured to emulate a different architecture from the at least one processor. For example, the virtual machine may be configured to emulate a quantum computing architecture on a silicon computer chip. Examples of virtual machines may include, but are not limited to, VMware®, VirtualBox®, Parallels®, QEMU®, Citrix® Hypervisor, Microsoft® Hyper-V®, or the like.

In the example disclosed in FIG. 2B, the system 200 further comprises a computing platform 204. The computational platform 204 may comprise a coherent optical network 222. The coherent optical network 222 may be of various types such as, for example, the types of coherent optical network as disclosed elsewhere herein.

The computational platform 204 may be operatively connected to the digital computer 202. The computational platform 204 may comprise a read-out control system 220. The read-out control system 220 may be configured to read information (e.g., computational results) from the at least one processing unit 218. For example and in FIG. 2A, the read-out control system may be configured to convert data from an FPGA to data usable by a digital computer.

The system may comprise a memory 206. The memory may be operatively connected to the digital computer 202. The memory 206 may be of various types. For instance and in some cases, the memory 206 comprises a database. Many types of databases may be suitable for storage and retrieval of data. In some cases, suitable databases comprise, by way of non-limiting examples, relational databases, non-relational databases, object-oriented databases, object databases, entity-relationship model databases, associative databases, and XML databases. In some cases, the database is Internet-based. In some cases, the database is web-based. In some cases, the database is cloud computing-based (e.g., on the cloud). In some cases, the database is based on one or more local computer storage devices. In some cases, solutions to solved problems are maintained by the database. In some cases, data indicative of a weighted maximum clique problem, data indicative of the stochastic differential equation, hyperparameters, a step size schedule, historical values of the partial derivatives, and batch sizes are stored in the database as well.

The computational platform 204 and the memory 206 may be connected to the digital computer 202 over a network. The computational platform, the memory, and/or the digital computer can have network communication devices. The network communication devices can enable the computational platform, the memory, and/or the digital computer to communicate with each other and with any number of user devices, over a network. The network can be a wired or wireless network. For example, the network can be a fibre optic network, Ethernet® network, a satellite network, a cellular network, a Wi-Fi® network, a Bluetooth® network, or the like. In other implementations, the computational platform, the database, and/or the digital computer may comprise several distributed computational platforms, databases, and/or digital computers that are accessible through the Internet. Such computational platforms, databases, and/or digital computers may be considered cloud computing devices. In some cases, the one or more processors of the at least one processor may be located in the cloud.

Still referring to FIG. 2, the computing system 200 may comprise a communications interface 216 for obtaining an indication of the stochastic differential equation and for providing at least one solution of the stochastic differential equation. The indication of the stochastic differential equation may be provided according to various examples disclosed herein. In some cases, the indication of the stochastic differential equation is provided by the digital computer to the communications interface 216 via a data network.

The communications interface 216 may be implemented according to various examples disclosed herein. In some cases, the communications interface 216 is implemented using an application programming interface (API) gateway configured to enable a user to transmit computational tasks and receive computational solutions.

In some cases, the application programming interface (API) gateway is programmed or configured to authenticate a user of the computing system. In some cases, the application programming interface (API) gateway is programmed or configured to monitor system and data security.

Now referring to FIG. 5, there is shown an example of a coherent optical network 222. The coherent optical network 222 may comprise a pulsed laser 502 for pumping to an optical cavity N times representative of N variables. The pumping comprises T repetitions. The coherent optical network 222 may comprise a plurality of beam splitters 504, 510, 512, 516, 518, 520 for splitting the laser beams into two or more parts and for combining two or more laser beams into a single pulse.

The coherent optical network 222 may comprise a degenerate optical parametric oscillator (OPO) comprising a second harmonic generation (SHG) 506 and a nonlinear amplifier 508.

The laser beams from the pulsed laser 502 may be split into two parts using the beam splitter 504, wherein one part is diverted to the beam splitter 516 and the second part is diverted to the second harmonic generation (SHG) 506. In some cases, reference pumps are generated from the SHG 506. The reference pumps from the SHG 506 are then conveyed to the nonlinear amplifier 508. In some cases, the reference pumps are conveyed in such a way that a pump for each variable reaches the nonlinear amplifier at the same time as the corresponding pump from the beam splitter 520 reaches the nonlinear amplifier 508. In some cases, the pulses from the beam splitter 504 sent to the beam splitter 516 are split by the beam splitter 516 into two parts, wherein one part is sent to the beam splitter 518 and the second part is sent to the beam splitter 512.

The nonlinear amplifier may comprise a cavity and a nonlinear crystal. The cavity and the nonlinear crystal create and amplify degenerate optical parametric oscillator (OPO) 524 pulses. The degenerate OPO 524 pulses are sent to the beam splitter 510. The degenerate OPO 524 pulses are split into two parts using the beam splitter 510. The coherent optical network may comprise a measurement feedback apparatus 522 comprising an electronic circuit 514. In some cases, one part of the degenerate OPO 524 pulses split in the beamer splitter 510 is used to feed the measurement feedback apparatus 522 whereas the other part is sent to the beam splitter 520 to meet the pulse that is sent from the beam splitter 518.

In the beamer splitter 512, the degenerate optical parametric oscillator (OPO) 524 pulses sent from the beam splitter 510 meet the pulses sent from the beam splitter 516; further, these pulses are combined into a single pulse which is diverted to the electronic circuit 514. In the electronic circuit 514, the optical information from the pulses is transformed into digital information to perform the calculations using a field-programmable gate array (FPGA). Next, the digital information corresponding to the results of the calculations is transformed back into optical information and is reinjected into the system using the beam splitter 518.

In the beam splitter 518, pulses sent from electronic circuit 514 and the pulses that are sent from the beam splitter 516 are combined, then the combined pulses are sent to the beam splitter 520. In the beam splitter 520, injection pulses sent from the beam splitter 518 are combined with the corresponding pulses sent from the beam splitter 510.

In some cases, the pulses are generated, split, transformed, combined, and sent as described herein until the system converges to an energy level.

### Computer Systems

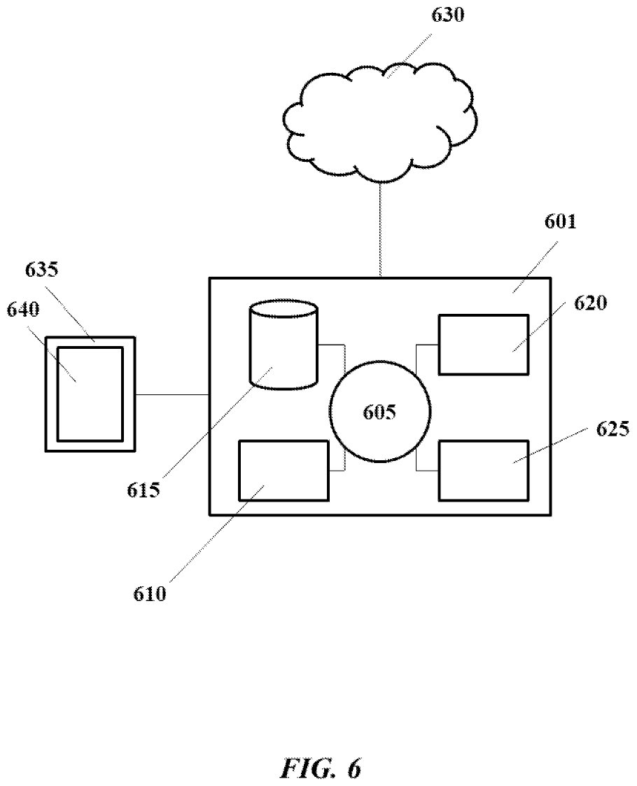

The present disclosure provides computer systems that are programmed to implement methods of the disclosure. FIG. 6 shows a computer system 601 that is programmed or otherwise configured to implement the methods disclosed elsewhere herein. The computer system 601 can regulate various aspects of the present disclosure such as, for example, to determine a solution for a weighted maximum clique problem. The computer system 601 can be an electronic device of a user or a computer system that is remotely located with respect to the electronic device. The electronic device can be a mobile electronic device. The computer system may be a computer or computational platform as described elsewhere herein.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, an interface configured to receive an indication of a weighted maximum clique problem. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, solve a weighted maximum clique problem.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for solving a weighted maximum clique problem, the method comprising:
(a) at an interface, obtaining an indication of said weighted maximum clique problem;
(b) formulating said weighted maximum clique problem as a quadratic continuous optimization problem comprising at least one simplex constraint;
(c) constructing a stochastic differential equation corresponding to said quadratic continuous optimization problem, wherein said stochastic differential equation comprises a plurality of continuous variables;
(d) using at least one of an optical computing device or a matrix multiplication device to solve said stochastic differential equation at least one time;
(e) at said interface, obtaining at least one solution of said stochastic differential equation from said optical computing device or said matrix multiplication device; and
(f) using said at least one solution of said stochastic differential equation to obtain at least one solution of said weighted maximum clique problem.

2. The method as claimed in claim 1, wherein (d) further comprises calculating at least one stochastic gradient and updating said plurality of said continuous variables.

3. The method as claimed in claim 2, wherein said updated plurality of continuous variables are implemented on said optical computing device to obtain an updated solution to said stochastic differential equation.

4. The method as claimed in claim 2, further comprising repeating (a) to (e) at least one time using said updated plurality of continuous variables.

5. The method as claimed in claim 1, wherein (f) comprises selecting a highest clique value.

6. The method as claimed in claim 1, wherein said interface is configured to communicate with a matrix multiplication device.

7. The method as claimed in claim 1, wherein said matrix multiplication device comprises at least one member selected from the group consisting of a graphics processing unit (GPU), a tensor processing unit (TPU), a field-programmable gate array (FPGA), an application-specific integrated circuit (ASIC), and a tensor streaming processor (TSP).

8. The method as claimed in claim 1, further comprising using said stochastic differential equation to configure said optical computing device.

9. The method as claimed in claim 1, further comprising using said stochastic differential equation to configure said matrix multiplication device.

10. The method as claimed in claim 1, wherein said optical computing device is a coherent optical network.

11. The method as claimed in claim 10, wherein said coherent optical network is a coherent Ising machine.

12. The method as claimed in claim 1, wherein said weighted maximum clique problem is a graph similarity problem or a molecular similarity problem.

13. The method as claimed in claim 1, wherein said interface is a cloud computing interface or a graphical user interface.

14. The method of claim 1, wherein said quadratic continuous optimization problem comprises a plurality of simplex constraints.

15. A system for solving a weighted maximum clique problem, the system comprising:
(a) a digital computer comprising an interface and a non-transitory computer readable medium operatively coupled to a processor, said non-transitory computer readable medium comprising instructions, wherein said processor is configured to execute said instructions to at least:
(i) obtain an indication of said weighted maximum clique problem,
(ii) formulate said weighted maximum clique problem as a quadratic continuous optimization problem comprising one simplex constraint, and
(iii) construct a stochastic differential equation corresponding to said quadratic continuous optimization problem, wherein said stochastic differential equation comprises a plurality of continuous variables;
(b) a memory operatively coupled to said digital computer, wherein said memory stores at least one solution of said stochastic differential equation and at least one solution of said weighted maximum clique problem; and
(c) a computational platform operatively coupled to said digital computer, said computational platform comprising at least one processor and a readout control system, wherein said computational platform is configured at least to:
(i) receive from said digital computer an indication of said stochastic differential equation,
(ii) solve at said at least one processor said stochastic differential equation to generate a solution, and
(iii) via said readout control, store said solution of said stochastic differential equation in said memory.

16. The system as claimed in claim 15, wherein said computational platform comprises at least one member selected from the group consisting of a field-programmable gate array (FPGA), an application-specific integrated circuit (ASIC), a central processing unit (CPU), a graphics processing unit (GPU), a tensor processing unit (TPU), a tensor streaming processor (TSP), an optical computing device, and a coherent optical network.

17. The system as claimed in claim 15, wherein one or more processors of said at least one processor is located on a cloud.

18. The system as claimed in claim 15, wherein said memory is operatively coupled to said digital computer over a network.

19. The method of claim 1, further comprising solving a graph similarity problem based at least in part on said at least one solution of said weighted maximum clique problem.

20. The method of claim 1, further comprising solving a molecular similarity problem based at least in part on said at least one solution of said weighted maximum clique problem.
